(19) United States
(12) Patent Application Publication
(10) Pub. No.: US 2021/0244958 A1
(43) Pub. Date: Aug. 12, 2021

(54) Primates Musculoskeletal Treatment Apparatus and Driving Method Thereof

(71) Applicant: Whi Young KIM, Busan (KR)

(72) Inventor: Whi Young KIM, Busan (KR)

(21) Appl. No.: 16/802,589

(22) Filed: Feb. 27, 2020

(30) Foreign Application Priority Data
Feb. 7, 2020 (KR) 10-2020-0014983

(51) Int. Cl. A61N 2/02

(52) U.S. Cl. CPC A61N 2/02

(57) ABSTRACT

An animal musculoskeletal treatment apparatus according to an embodiment of the present invention includes: a stimulation coil unit outputting a magnetic stimulation pulse as a treatment pulse for a predetermined lesion; a stimulation pulse driving unit generating and providing, to the stimulation coil unit, treatment pulses with different frequencies according to a type of a lesion; and a controller controlling the stimulation pulse driving unit such that the generated treatment pulses with different frequencies are selectively output to the stimulation coil unit.

### BACKGROUND OF THE INVENTION

### Field of the Invention

The present invention relates to an animal musculoskeletal treatment apparatus and a driving method thereof and, more particularly, to an animal musculoskeletal treatment apparatus that promotes regeneration and recovery of muscles such as skin, muscles, ligaments, and the skeleton when they are damaged because they are regenerated from bone marrow mesenchymal cells and muscle satellite cells but it is slow and inefficient, and a driving method thereof.

### Description of the Related Art

In muscle fibers, mononuclear fibrillary cells are classified in accordance with fibrillary myoblastic stages, propagate in sarcolemmas along necrosis parts, are positioned between sarcoplasms and basilar membranes after separating from the muscle fiber, and propagate into mononuclear fibrillary cells through mitosis. The reason that a sacroplasm is basophilic in the early stage of regeneration is because there is sufficient RNA in fibrillary cellularities, pseudopodia of sarcroplastic buds grow at ends of muscle fibers injured during fusion, myotube, and formation of mononuclear fibrillaries and are connected to another injured part, fibrillary cells cover the surfaces of the pseudopodia, the band-shaped fibrillary cells are surrounded by micronuclear sarcoplasms, myofibrils are observed around myonuclei, and mitosis no longer occur after connection with another myofibril.

While a myotube changes into a matured muscle fiber, a myonucleus moves around, and a myofilament becomes thick and long into a myofibril, an increasing mechanism is similar to myogenesis in the generation stage, and change, regeneration, growth, and adjustment of a myogenic regulatory factor (MRF) in-muscle fiber phenotype require a change of factors such as the concentration of mRNAs and DNAs in a muscle fiber, an increase in protein composite ratio, an oxidative metabolic ability, troponin I, and sarcoplasmic Ca+ dependent ATPase, in which a myogenic regulatory factor (MRF) that is the transcription factor in a nucleus is in associated. The MRF is classified into MyoD, Myf-5, myogenin, and MRF-4 and has a bHLH (basic helix-loop-helix) region, MRF revelation causes growth of a muscle fiber, in-nucleus activity in a satellite cell, latency, growth, and change in an inactive state between a basilar membrane and a cell membrane of a muscle fiber as a myogenic precursor cell of a skeletal muscle fiber, active change and latency, dissolution in a muscle fiber, and is transcribed in the nucleus of the satellite cell, and the MyoD and the miogenin, which are control factors in a muscle fiber, are not revealed in a normal muscle cell, and the amount of protein increases for a regeneration period after muscle injury.

Muscle injury causes contraction and oxidative stress such as heat generation in a skeletal muscle in motion due to stimulation, production of reactive oxygen species (ROS), exhaustion of an energy source deteriorates homeostasis in a skeletal muscle and structurally and biochemically injures muscle cells.

### Documents of Related Art

(Patent Document 0001) Korean Patent No. 10-0924984 (2009, Oct. 28)

(Patent Document 0002) Korean Patent No. 10-1148096 (2012, May 15)

(Patent Document 0003) Korean Patent No. 10-1580036 (2015, Dec. 17)

(Patent Document 0004) Korean Patent No. 10-1695096 (2017, Jan. 4)

(Patent Document 0005) Korean Patent No. 20-0428468 (2006, Oct. 2)

(Patent Document 0006) Korean Utility Model Application Publication No. 20-2019-0001779 (2019, Jul. 10)

### SUMMARY OF THE INVENTION

An embodiment of the present invention provides an animal musculoskeletal treatment apparatus that promotes regeneration and recovery of muscles such as skin, muscles, ligaments, and the skeleton when they are damaged because they are regenerated from bone marrow mesenchymal cells and muscle satellite cells but it is slow and inefficient, and a driving method thereof.

An animal musculoskeletal treatment apparatus according to an embodiment of the present invention includes: a stimulation coil unit outputting a magnetic stimulation pulse as a treatment pulse for a predetermined lesion; a stimulation pulse driving unit generating and providing, to the stimulation coil unit, treatment pulses with different frequencies according to a type of a lesion; and a controller controlling the stimulation pulse driving unit such that the generated treatment pulses with different frequencies are selectively output to the stimulation coil unit.

The animal musculoskeletal treatment apparatus may further include an electromyogram sensor, in which the controller may control the stimulation pulse driving unit to generate a treatment pulse for a predetermined lesion further in consideration of a sensing value of the electromyogram sensor.

The stimulation pulse driving unit may use a multiple discharge method of outputting treatment pulses by sequentially generating pulses in a pulse sequence type for a predetermined lesion.

The stimulation pulse driving unit may output a treatment pulse by adjusting pulse intensity, a pulse shape, and a pulse width in accordance with a delay time on the basis of the multiple discharge method.

The stimulation pulse driving unit may configure a pulse forming circuit composed of a resistor, a capacitance, and an inductance to supply energy to the stimulation coil unit, and the pulse forming circuit may configure a multiple L-C network.

A method of driving an animal musculoskeletal treatment apparatus according to an embodiment of the present invention includes: outputting a magnetic stimulation pulse as a treatment pulse for a predetermined lesion by means of a stimulation coil unit; generating and providing, to the stimulation coil unit by means of a stimulation pulse driving unit, treatment pulses with different frequencies according to a type of a lesion; and controlling the stimulation pulse driving unit such that the generated treatment pulses with different frequencies are selectively output to the stimulation coil unit by means of a controller.

The method may further include measuring a state of a muscle by means of an electromyogram sensor, in which the controlling may control the stimulation pulse driving unit to generate a treatment pulse for a predetermined lesion further in consideration of a sensing value of the electromyogram sensor.

The providing of treatment pulses to the stimulation coil unit may use a multiple discharge method of outputting treatment pulses by sequentially generating pulses in a pulse sequence type for a predetermined lesion.

The providing of treatment pulses to the stimulation coil unit may output a treatment pulse by adjusting pulse intensity, a pulse shape, and a pulse width in accordance with a delay time on the basis of the multiple discharge method.

The stimulation pulse driving unit may configure a pulse forming circuit composed of a resistor, a capacitance, and an inductance to supply energy to the stimulation coil unit, and the pulse forming circuit may configure a multiple L-C network.

In an embodiment of the present invention, in influence on blood vessels by a magnetic field, contraction and stretching of arterioles and capillaries are directly generated by stimulating sympathetic nerves and a sympathetic signal transmitted to the brain through the spinal cord influences contraction of blood vessels of skin sympathetic nerves and activity of expansion fibers, thereby being able to increase the amount of blood in the capillaries and the blood vessels.

Further, an embodiment of the present invention mediates between cell growth accelerator and cytokine, propagates a fibrillary cell, forms a myotube cell, promotes activation of a satellite cell, and makes a base for muscle regeneration. When magnetic stimulation is supplied to blood flow as a related factor in an osteoblast differentiation and activation in the bone (tissue) of a skeleton, a cartilage, a ligament, and a skeletal muscle, red corpuscles are separated, the mechanism of action of the blood becomes active, the blood circulation is normalized by promoting an ion effect, oxygen efficiency is increased, and activation of osteoblasts us promoted.

Further, in an embodiment of the present invention, a stimulation pulse driving unit (analog part), a microcontroller unit, and a Bluetooth module are configured, and a system is simply configured to implement a stimulation pulse, thereby being able to not only influence propagation and differentiation of osteocytes and recovery and regeneration of osseous tissues, but also increase activation of skeletal tissue cells such as a skeletal ligament cell and a skeletal fibroblast and clinically regenerate a lost skeletal tissue.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a diagram showing the entire configuration of an animal musculoskeletal treatment apparatus according to an embodiment of the present invention;

FIG. 2 is a view showing a driving process of the animal musculoskeletal treatment apparatus shown in FIG. 1;

FIG. 3 is a view showing a treatment process using the animal musculoskeletal treatment apparatus shown in FIG. 1;

FIG. 4 is a view showing a promotion process of an osteoclast and an osteoblast;

FIG. 5 is a circuit diagram forming a multi-stage L-C network pulse;

FIG. 6 is an exemplary view of the operation of the circuit diagram forming a multi-stage L-C network pulse shown in FIG. 5;

FIGS. 7 to 9 are views showing a process for treatment, communication, control, and connection with an associated person using a cell phone; and

FIG. 10 is a flowchart showing a driving process of an animal musculoskeletal treatment apparatus according to an embodiment of the present invention.

### DETAILED DESCRIPTION OF THE INVENTION

Hereinafter, embodiments of the present invention will be described in detail with reference to the accompanying drawings.

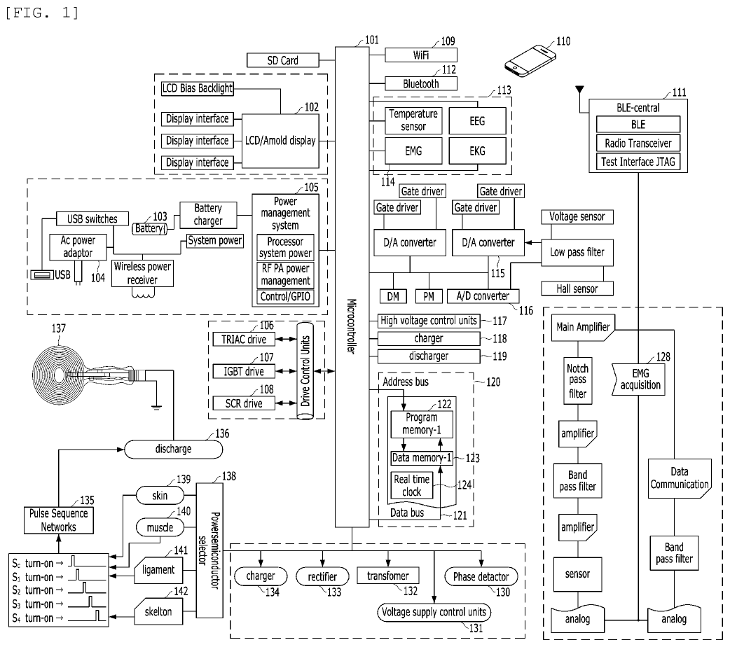

FIG. 1 is a diagram showing the entire configuration of an animal musculoskeletal treatment apparatus according to an embodiment of the present invention.

As show in FIG. 1, an animal musculoskeletal treatment apparatus 90 according to an embodiment of the present invention is designed to perform various treatment processes in connection with applications (hereafter, Apps) using pulse forms for example according to the muscular skeleton of an animal and includes some or all of: a controller 101, a user interface 102, a power supplier 103˜105, a driver 106˜108, a communication unit 109˜112, a sensor unit 113, 114, a peripheral circuit 115˜119, a memory 120˜124, an EMG amplifier 125, 128, a high-voltage generator 130˜134, an a stimulation pulse driver (or a treatment pulse generator) 135˜142.

The term ‘includes some or all” means that the animal musculoskeletal treatment apparatus 90 is configured without some components such as the memory 120˜124 or some components such as the driving driver 106˜108 are included in other components such as the controller 101, and it is exemplified in the following description that all of them are included to help sufficiently understand the present invention.

The controller 101 controls the general operations of various components of the animal musculoskeletal treatment apparatus 90. The controller includes a microprocessor, which is an important part of the animal musculoskeletal treatment apparatus 90, and the microprocessor may include a CPU, an MPU, or the like.

The user interface 102 includes a display including a device such as an LCD or an OLED and may further include a power button, etc. The display may include a touch panel, thereby being able to receive instructions from a user. Images are implemented in the monitor of the display and various items of information can be displayed in the images.

The power supplier 103˜105 may include an adaptor for using LCD supply voltage, a built-in charger, a power battery 103 for use of a mobile phone, an AC power adapter 104, a power manager 105 controlling a power function of the microprocessor, etc. The power manager 105 can examine the remaining power of the battery 103.

The driver 106˜108, for example, can take part in control of the stimulation pulse driver 135˜142 and may include a TRIAC driver 106, an IGBT driver 107, and an SCR driver 108.

The communication unit 109˜112 includes a first communication unit 109 performing WiFi communication, a cell phone 110 performing various treatment processes through Apps therein associated with the animal musculoskeletal treatment apparatus, a second communication unit 111 performing Bluetooth communication for data processing, and a (communication) port 112 processing data received from the microprocessor through Bluetooth.

The sensor unit 113, 114 includes various application sensor ports 113 and a muscle conduction sensor 114.

The peripheral circuit 115˜119 includes a D/A converter 115 that converts a digital signal into an analog signal, an A/C converter 116 that converts an analog signal into a digital signal, a high-voltage controller 117 that is recognized by a processor, a charger 118 that is recognized by the processor, and a discharger 119 that is recognized by the processor. The peripheral circuit 115˜119 can be operated under the control of the controller 101.

The memory 120˜124 includes an address bus 120, a data bus 121, a program memory 122, a data memory 123, a real-time clock generator 124, etc. The real-time clock generator 124, for example, can be used to give notice of start of data processing.

The EGM amplifier 125, 128 includes an A/D converter 125 that converts a an analog signal into a digital signal for muscle conduction and obtained muscle conduction data are transmitted to a cell phone or a microprocessor through Bluetooth 128.

Reference numerals 130˜137 indicate hardware of the animal musculoskeletal treatment apparatus. The high-voltage generator 130˜134 includes a phase detector 130 that detects the phase of power that is obtained from AC power, a voltage supply controller 131, a transformer 132 that increases the voltage of use power, a rectifier 133 that converts an increased AC voltage into a DC voltage, and a charger 134.

The stimulation pulse driver 135˜142 includes a pulse generator 135 that sequentially forms and controls pulses in a pulse sequence conception, a discharger 136 that discharges prepared voltage, and a (stimulation) coil 137 that operates a treatment pulse as the function of a pulse sequence, and functions as a final load. The stimulation pulse driver may refer to the part excluding the stimulation coil 137.

The stimulation pulse driver 135˜142 selects supply energy (e.g., stimulation pulses with different frequencies) in accordance with a skin, muscle power, a ligament, and a skeleton using a power semiconductor selector 138, thereby implementing optimal treatment in accordance with lesions such as a skin 139, muscle power 140, a ligament 141, and a skeleton 142.

Muscle injury causes a problem with muscle activity, a loss of muscular skin, and a lack of exercise in humans and animals, thereby not only causing a loss of active ability, but also influencing the quality of life of human and animals. Muscle injury is severe when eccentric contraction occurs rather than concentric contraction or isometric contraction, Skeletal muscles are tissues that are sensitive to stimulation, and oxidative stress such as heat generation in a skeletal muscle in motion due to stimulation, production of reactive oxygen species (ROS), exhaustion of an energy source deteriorates homeostasis in a skeletal muscle and structurally and biochemically injures muscle cells. A physiological/biochemical change of a muscle cell causes disruption of a myofibril, injury of a cell membrane, production of substances related to edema and apoptosis, thereby progressing muscle injury, reducing the ability to generate power of a skeletal muscle, causing delayed onset muscle soreness (DOMS). Further, the physiological/biochemical change is caused by a genetic disorder, cancer, inflammation, infection or various medical influences.

Accordingly, in an embodiment of the present invention, the animal musculoskeletal treatment apparatus 90 shown in FIG. 1 promotes regeneration and recovery of muscles such as skin, muscles, ligaments, and the skeleton when they are damaged because they are regenerated from bone marrow mesenchymal cells and muscle satellite cells but it is slow and inefficient.

In more detail, muscle regeneration includes a process of necrosis, degeneration, inflammation, regeneration, and fibrosis, and excessive force increase a sarcomere length out of a normal contraction range, so large tension injures muscle tissues. Further, cytoskeletons have an important role in forming sacomere tissues and cell membranes. Destruction of a muscle fiber destructs a muscle tissue, a blood-borne inflammatory cell is sent to an injured portion, and MD immune cells such as a neutrophil and a macrophage remove tissue sections, etc., and greatly contribute to regenerating injured muscles but delay recovery. In particular, magnetic stimulation may be considered as bone coupling, pain mitigation, and soft tissue swelling;

treatment of a fracture including nonunion, arthrosis, necrosis of bone, chronic obstinate tenosis in bone coupling, and reduction of swelling in injury of the muscular skeleton, post operative injury, posttraumatic injury, chronic injury reduce a pain, recover and regenerate nerves, and increase an immune function, an endocrine function, and fundamental functions. Accordingly, when magnetic stimulation is supplied to blood flowing in osseous tissues, the iron in the red corpuscles is influenced, so conglomerated red corpuscles are separated, the mechanism of action of the blood becomes active, the blood circulation is normalized by promoting an ion effect, oxygen efficiency is increased, and activation of osteoblasts are promoted. An implant bone adhesion stimulation apparatus that uses magnetic pulses for early settlement after implanting related to the bone (tissue) of a skeleton, a cartilage, a ligament, and a skeletal muscle may be provided.

TNF-α (tumor necrosis factor-α) and IL-1β that are proinflammatory cytokines recognize a tissue injury and act on blood cell, revelation of neutrophil granulocyte, chemoattractant of the injured portion and IL-6 (interleukin-6) and IL-8 (interleukin-8) that are proinflammatory cytokines are secreted, MPO (myeloperoxidase), NADPH (nicotinamide adenine dinucleotide phosphate), etc. are returned into ROS (reactive oxygen species), a respiratory burst destructs injured muscle tissues and performs phagocytosis, hydrogen peroxide and hydroxyl radical injure a myotube cell and eaten by a macrophage, and lysosomal protease and NO (nitric oxide) such as cathepsin B, L, H, and C are secreted. The macrophage mediates between cell growth accelerators, such as FGF (fibroblast growth factor), IGF-1, and TGF-β1 (transforming growth factor-β1), and cytokine, propagates a fibrillary cell, forms a myotube cell, promotes activation of a satellite cell, and makes a base for muscle regeneration. Further, when magnetic stimulation is supplied to blood flowing in a parathyroidhormone osteocyte as a related factor in an osteoblast differentiation and activation in the bone (tissue) of a skeleton, a cartilage, a ligament, and a skeletal muscle, the iron in the red corpuscles is influenced, so conglomerated red corpuscles are separated, the mechanism of action of the blood becomes active, the blood circulation is normalized by promoting an ion effect, oxygen efficiency is increased, and activation of osteoblasts are promoted. The magnetic stimulation not only influences propagation and differentiation of osteocytes and recovery and regeneration of osseous tissues, but also increases activation of skeletal tissue cells such as a skeletal ligament cell and a skeletal fibroblast and can be clinically used in regeneration of a lost skeletal tissue.

To this end, in an embodiment of the present invention, a stimulation pulse driving unit (analog part), a microcontroller unit, a Bluetooth module, etc. are configured and a system is configured to implement a stimulation pulse.

Muscle injury causes contraction and oxidative stress such as heat generation in a skeletal muscle in motion due to stimulation, production of reactive oxygen species (ROS), and exhaustion of an energy source deteriorates homeostasis in a skeletal muscle and structurally and biochemically injures muscle cells.

Accordingly, in an embodiment of the present invention, operation of an osteoclast is promoted in an inflammation stage through a magnetic stimulation pulse and an osteoblast further promotes the operation, thereby reducing the treatment period.

In destruction of a muscle fiber and a muscle tissue, an inflammatory cell is sent to an injured portion, thereby increasing the active level of a neutrophil and a macrophage, tissue sections of immune cells are removed, injured cells are removed, recovered, and regenerated, TNF-α and IL-1β that are proinflammatory cytokines promote secretion, act on a vascular endothelial cell, promote revelation of P-selectin and E-selectin in neutrophil adhesion, and activate secretion of IL-6 and IL-8, a neutrophil returns into reactive oxygen species (ROS) such as MPO and NADPH, a pseudopodia operates for eating due to promotion of activity of a macrophage, mediation of FGF, IGF-1, TGF-β1, and factors promoting cell growth, and proinflammatory cytokine is performed, a fibrillary cell is propagated, a myotube cell is formed, activation of a satellite cell is promoted, and muscle regeneration is promoted.

In an embodiment of the present invention, muscle diagnosis is required in real time through magnetic stimulation and a (electromyogram) sensor, for accurate diagnosis and treatment of a lesion, EMG and appropriate stimulation treatment is performed, emergency prescription by a doctor is added to the sensor and the stimulation coil through real-time wire/wireless communication, depending on the condition of symptoms, and real-time monitoring and stimulation treatment can be achieved through a cell phone, etc.

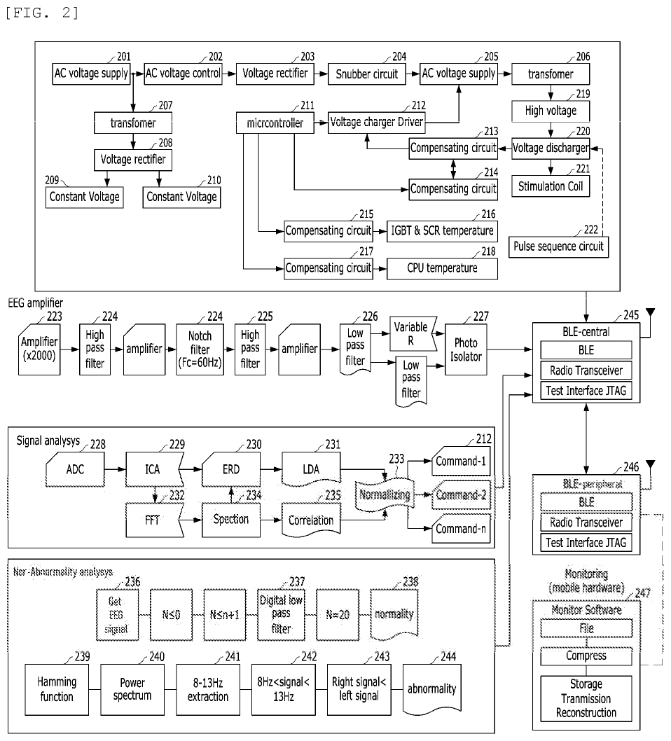

FIG. 2 is a view showing a driving process of the animal musculoskeletal treatment apparatus shown in FIG. 1.

Reference numerals 201 to 222 indicate the operation of the animal musculoskeletal treatment apparatus, reference numerals 227 to 236 indicate the operation of an EMG amplifier, and reference numerals 237 to 250 indicate a process of processing obtained data signals.

First, the operation of the animal musculoskeletal treatment apparatus is described. The operation is performed by a voltage supplier 201 that supplies AC power, an AC voltage control circuit 202, a voltage rectifier 203, a snubber circuit 204, a charging voltage function 205, a transformer 206 that amplifies voltage in proportion to a winding ratio, a transformer 207 that decrease voltage in proportion to a winding ratio, a function of rectifying voltage coming out of the transformers 208, a DC 16V constant voltage circuit 209, a DC 5V constant voltage circuit 210, a microprocessor 211, a voltage charging driving circuit 212, an compensation circuit 213, a compensation circuit 214 recognized by the microprocessor 211, an IGBT, an SCR temperature measurement compensation circuit 215, an IGBT, an SCR temperature measurement function 216, a compensation circuit 217 recognized by the microprocessor 211, a CPU temperature measurement function 218, a high-voltage circuit 219, a voltage discharge function 220, a treatment stimulation coil 221, a pulse sequence driving circuit 222, etc.

Reference numeral 223 indicates a signal processing process. Reference numeral 224 indicates Bluetooth for receiving data obtained from electromyogram, reference numeral 225 indicates Bluetooth for reception in a cell phone, etc., and reference numeral 226 indicates a screen shown through a GUI by mobile hardware.

Next, the operation of the EMG amplifier is described. The operation is performed by an analog signal 228 in an electromyogram amplifier, a digital signal 229 in an electromyogram amplifier, an assistant sensor 230, a band-pass filter 231, a notch filter 232 that blocks a specific frequency, an A/D converter 233, data communication processing 234, a main amplifier 235, generally obtained electromyogram signal data 236, etc.

Further, the data signal processing process is performed by a converter 237 that converts an analog signal into a digital signal, ICA (Independent Component Analysis) 238, ERD (event related desynchronization) 239, LDA (discriminant analysis) 240, a processing command 241, generalizing (or normalizing) 242, FFT (Fast Fourier Transform) 243, a spectrum 244, correlation 245, a band-pass filter 246, a full-wave rectifier 247, a waveform detector 248, an adoptive comparer 249, a photo coupler 250 that is sent in an insulation state without a loss, etc.

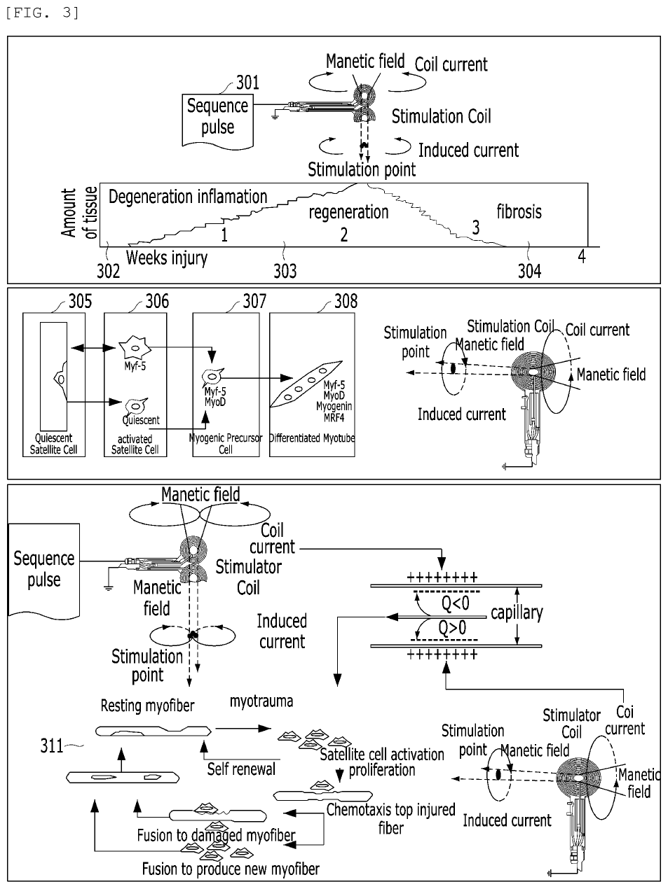

FIG. 3 is a view showing a treatment process using the animal musculoskeletal treatment apparatus shown in FIG. 1.

Referring to FIG. 3, muscle regeneration is circulated through necrosis, degeneration of reference numeral 302, inflammation, regeneration of reference numeral 303, and fibrosis of reference numeral 304, and the animal musculoskeletal treatment apparatus 90 according to an embodiment of the present invention has an effect that can reduce this process.

A muscle fiber, muscle tissue destruction, and blood-borne inflammatory cell are sent to an injured portion, immune cells such as a neutrophil and a macrophage remove tissue sections, etc., and greatly contribute to remove, recover, and regenerate injured cells, and TNF-α and IL-1β are secreted after recognizing an injury, act on a vascular endothelial cell, cause neutrophil granulocyte adhesion, and induce revelation of Pselectin and E-selectin that are adhesion molecules.

A muscle injury promotes a chemical action and activates secretion of chemotactic factors and IL-6 and IL-8 that are proinflammatory cytokines, neutrophil granulocyte returns to reactive oxygen species (ROS) such as MPO and NADPH, a respiratory burst destructs injured muscle tissues and performs phagocytosis, hydrogen peroxide and hydroxyl radical injure a myotube cell, byproducts in a muscle is eaten by a macrophage, and lysosomal protease and NO (nitric oxide) such as cathepsin B, L, H, and C are secreted.

The macrophage eats byproducts with pseudopodia, surrounds the periphery, removes endoplasmic reticulum, increases a muscle injury, mediates between proinflammatory cytokine and factors promoting cell growth such as FGF, IGF-1, and TGF-β1, propagates a fibrillary cell, forms a myotube cell, promotes activation of a satellite cell, and promotes a basis for muscle regeneration in accordance with an embodiment of the present invention as indicated by reference numeral 301.

A base membrane surrounding a muscle fiber is possible when a blood vessel and a satellite cell of reference numeral 305 exist, muscle regeneration occurs when a satellite cell of reference numeral 306 acts, desmin is revealed before a myotube and a myofilament are formed in muscle production, an adjustment factor in a muscle production process, the muscular satellite cell of reference numeral 311 starts acting with appearance of muscle fibers showing desmin positive immune reaction, a nucleus condensation phenomenon, two or more nuclei, metamophosis, and regeneration periods are classified by myoblastic stages, mononuclear fibrillary cells propagate in sarcolemmas along necrosis parts, the reason that a sacroplasm is basophilic in the early stage of regeneration is because there are sufficient RNAs in fibrillary cellularities, pseudopodia of sarcroplastic buds grow at ends of muscle fibers injured during fusion, myotube, and formation of mononuclear fibrillaries and are connected to another injured part, fibrillary cells cover the surfaces of the pseudopodia, while a myotube changes into a matured muscle fiber, a myonucleus moves around, and a myofilament becomes thick and long into a myofibril, an increasing mechanism is similar to myogenesis in the generation stage, and change, regeneration, growth, and adjustment of a myogenic regulatory factor (MRF) in-muscle fiber phenotype of reference numeral require a change of factors such as the concentration of mRNAs and DNAs in a muscle fiber, an increase in protein composite ratio, an oxidative metabolic ability, troponin I, and sarcoplasmic Ca+ dependent ATPase, in which a myogenic regulatory factor (MRF) that is the transcription factor in a nucleus is in associated.

The MRF is classified into MyoD, Myf-5, myogenin, and MRF-4 of reference numeral 308 and has a bHLH (basic helix-loop-helix) region, MRF revelation causes growth of a muscle fiber, in-nucleus activity in a satellite cell, latency, growth, and change in an inactive state between a basilar membrane and a cell membrane of a muscle fiber as a myogenic precursor cell of a skeletal muscle fiber, and MRF is transcribed in the nucleus of the satellite cell, and the MyoD and the miogenin, which are control factors in a muscle fiber, and the amount of protein increases for a regeneration period after muscle injury.

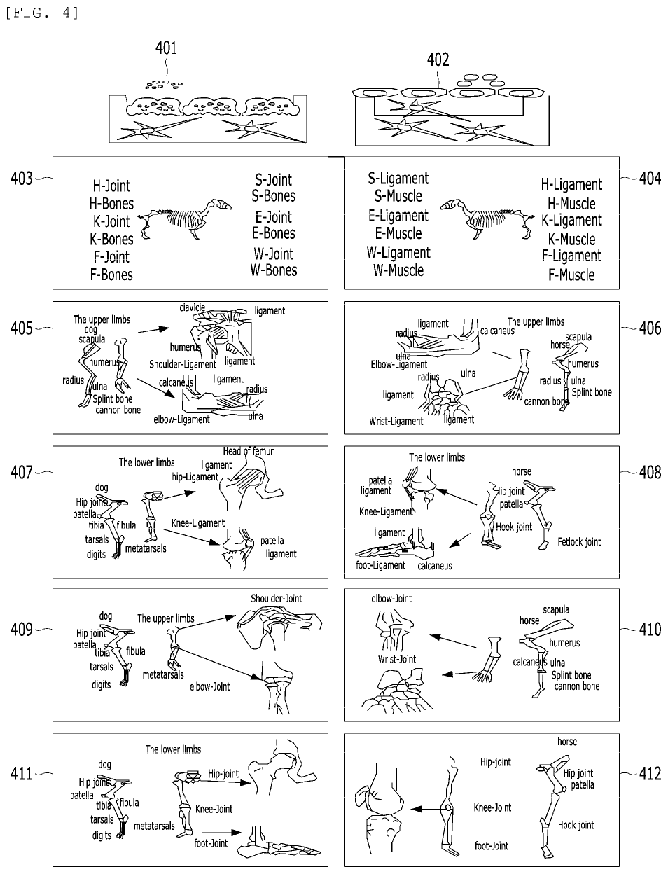

FIG. 4 is a view showing a promotion process of an osteoclast and an osteoblast.

Referring to FIG. 4, reference 401 promotes the operation of an osteoclast (blood, muscle, ligament) and reference number 402 further promotes operation of an osteoblast (blood, muscle, ligament).

In muscle regeneration, the period of necrosis, degeneration, inflammation, regeneration, and fibrosis is reduced, the active level of a neutrophil and a macrophage, TNF-α and IL-1β that are proinflammatory cytokines are secreted, and revelation of P-selectin and E-selectin that are adhesion molecules is induced and promoted.

A muscle injury promotes a chemical action and activates secretion of chemotactic factors and IL-6 and IL-8 that are proinflammatory cytokines, neutrophil granulocyte returns to reactive oxygen species (ROS) such as MPO and NADPH, a respiratory burst destructs injured muscle tissues and performs phagocytosis, hydrogen peroxide and hydroxyl radical injure a myotube cell, byproducts in a muscle is eaten by a macrophage, and lysosomal protease and NO (nitric oxide) such as cathepsin B, L, H, and C are secreted.

The macrophage eats byproducts with pseudopodia, surrounds the periphery, removes endoplasmic reticulum, increases a muscle injury, mediates between proinflammatory cytokine and factors promoting cell growth such as FGF, IGF-1, and TGF-β1, propagates a fibrillary cell, forms a myotube cell, promotes activation of a satellite cell, and promotes a basis for muscle regeneration.

Reference numeral 403 is effective for a bone and a joint, reference numeral 404 is effective for a muscle and a ligament, reference numeral 405 is effective for a thighbone, a ligament, etc., reference numeral 406 is effective for a lower limb bone, a ligament, etc., reference numeral 407 is effective for the joint of a lower limb, reference numeral 408 is effective for the ligaments of a lower limb, reference numeral 409 is effective for joints such as a shoulder and an elbow, reference numeral 410 is effective for wrist joints and bones, reference numeral 411 is effective for the hip joint, the knee joint, and the foot joint of a lower limb, and reference numeral 412 is effective for lower limb joints and knee caps.

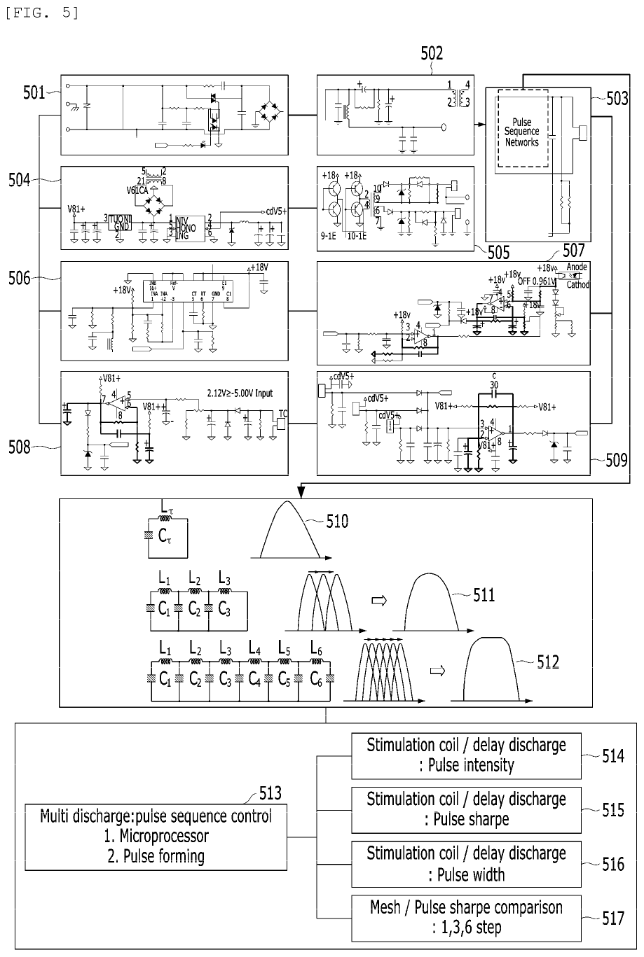

FIG. 5 is a circuit diagram forming a multi-stage L-C network pulse.

Referring to FIG. 5, the principle of a multi-stage L-C network pulse forming circuit is to form an output pulse from a treatment coil pulse by discharging a coil and to overlap several stages to obtain a current shape with flat tops through a treatment coil and an L-C circuit with harmonic impedance, in which energy stored in a capacitor of the network is entirely discharged and a desired pulse shape is obtained by appropriately delaying or limiting the discharge current using an inductor. In order to supply energy to a stimulation coil, a pulse forming network composed of a resistor R, a capacitance C, and an inductance L should be configured and magnetic stimulation operates as a single or multiple mesh network. These networks have a desired current pulse when storing and transmitting discharge energy to a coil, etc., when discharging is inappropriate, a countercurrent flows to the coil, etc., so the lifespan of the coil, etc. decreases and the discharge efficiency also decreases. Accordingly, the current pulse flowing through the coil, etc., should be critically damped, the stimulation coil is driven by a signal mesh or multiple mesh LC network, coil input energy Eo, a pulse width tp, and a coil parameter should be determined before PFN design, a signal mesh PFN generates a sine wave pulse, a multiple mesh RFN generates a square wave pulse of which the peak power and the average power are almost the same, and the pulse shapes should be determined in accordance with applications. The multiple mesh is very interesting in terms of using, the number of meshes is increased in accordance with the same pulse width and sine waves that are peak values, so the pulse width is inversely proportioned to the number of meshes with the peak value maintained.

As the number of meshes is increased, the rising time and the falling time of square waves become short. In particular, rapid rising time sputtering is caused, so attention is required when determining the number of meshes. In the present invention, the PFN is configured into one-stage, 3-state, and 6-stage meshes, and in order to obtain stable output without a loss, a capacitance CT value, an inductance LT value, and the charge voltage of the capacitance that are (critical braking) circuit integers are considered.

A method according to an embodiment of the present invention is pulse shape control using a multiple discharge method, in which a treatment stimulation coil is discharged in consideration of pulse intensity, a pulse shape, and a pulse with in accordance with delay time, and the number of meshes of 1-stage, 3-stage, and 6-stage are compared in comparison according to pulse shapes. Reference numeral 501 is a common power device that can adjust AC power, reference numeral 502 is a DC high-voltage unit generating DC through a bridge diode, reference numeral 503 is a pulse sequence network, reference numeral 504 is a constant voltage circuit after AC voltage is decreased, reference numeral 505 is an amplification circuit for driving an IGBT, reference numeral 507 is a PWM circuit for driving the IGBT, reference numeral 508 is a low-band filter circuit, reference numeral 509 is a low-band filter limit circuit, reference numeral 510 is a low-band filter limit circuit having an integration function, reference numeral 511 is a 1-stage pulse sequence circuit, reference numeral 512 is a 3-stage pulse sequence circuit, and reference numeral 513 operates as a 5-stage pulse sequence circuit. Other detailed connection relationships of circuits refer to FIG. 5.

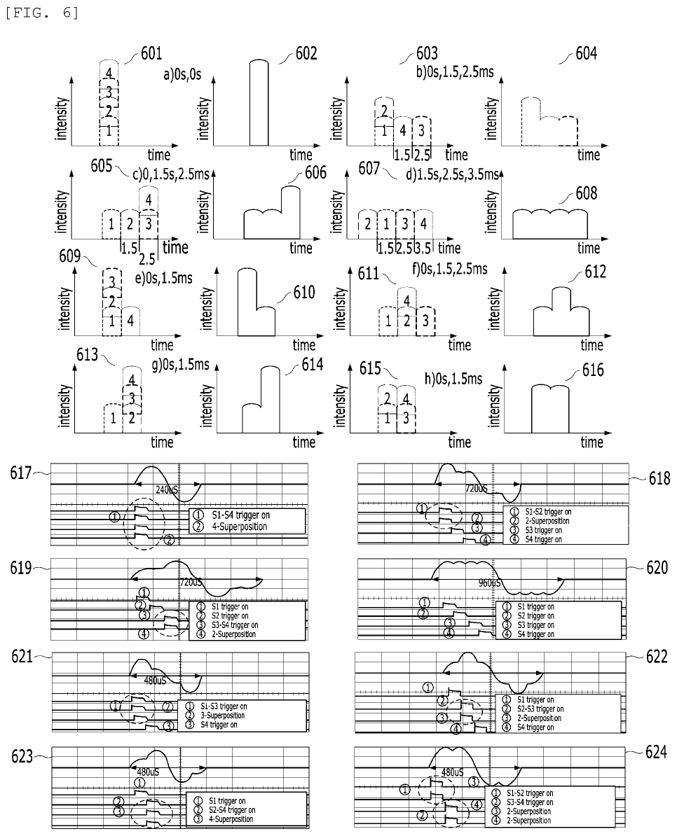

FIG. 6 is an exemplary view of the operation of the circuit diagram forming a multi-stage L-C network pulse shown in FIG. 5.

A muscle regeneration activity goes through necrosis/degeneration, inflammation, regeneration, and fibrosis processes, an injured muscle is recovered by an increase in the number, length, and thickness of fibers after injured, adjustment is performed by muscle gene in the regeneration process, a cell period adjustment mechanism is adjusted for propagation and differentiation of a myogenic precursor cell, MyoD starts differentiation and activates transcription by bonding to the promoters of myogenin and MRF in nuclei and MEF2C and pRB as a transcription factor. A regeneration process such as protein synthesis and degeneration reduction is accelerated, a muscle pain is reduced, muscle regeneration is promoted, and the revelation amount of protein of MyoD and myogenin is increased after muscle injury. As a result, it can be seen that magnetic stimulation accelerates muscle regeneration by activating myogenin through MyoD.

It can be seen that magnetic stimulation regenerates skeletal muscles, promotes regeneration of injured muscles, and activates AKT (protein kinase B)/mTOR (mammalian target of rapamycin) signal transmission paths that prevent a loss due to inactivation, muscle differentiation and marrow mesenchymal cells are converted into muscle cells by MyoD, a muscle regeneration mechanism prevents DNA methylation, performs antioxidation, and has an effect in gene adjustment for muscle cell adjustment, and muscle regeneration can activate AKT/mTOR signal transmission paths.

Discharging of a multiple L-C network is performed after energy is stored in a capacitor in which the structure of the capacitor should be larger than an allowable current, and in order to change the energy per pulse and a pulse width after an operation voltage is determined, capacitance and inductance should be changed and treatment coil pulse variables should be freely changed.

A thyristor, an SCR pair, and a GTO (gate turn off) that are used as switching elements of a main discharge circuit are output pulse formation methods developed more than pulse formation by an L-C circuit and can continuously adjust output pulse widths. These methods have a defect that control is difficult and it is difficult to adjust the output intensity in a single pulse.

When SCR is turned on, energy is sufficiently stored in a capacitance of a PFN, SCRs S1, S2, and S3 are sequentially turned on with predetermined delay times, and the energy stored in the capacitance of the PFN is transmitted to a treatment coil, whereby operation is performed. Reference numeral 610 is a state without a delay time, reference numeral 617 is also a experiment waveform without a delay time, reference numeral 603 is 0, 1.5, 2.5 mS, reference numeral 605 is 0, 1.5, 2.5 mS, reference numeral 607 is 1.5, 2.5, 3.5 mS, reference numeral 609 is 0, 1.5 mS, reference numeral 615 is 0, 1.5 mS, reference numeral 618 is the same principle as reference numeral 603, reference numeral 619 is the experiment waveform of reference numeral 605, reference numeral 620 is the experiment waveform of reference numeral 607, reference numeral 621 is the experiment waveform of reference numeral 609, reference numeral 622 is the experiment waveform of reference numeral 611, reference numeral 623 is the experiment waveform of reference numeral 613, and reference numeral 624 the experiment waveform of reference numeral 615.

The principle is to control a turn-on delay time of an SCR configured as a microprocessor using a control circuit, the control circuit is composed of a keyboard for inputting a delay time for operation, FNDs (Multisegmented LED Displays) displaying the delay time, a microprocessor that is the most important part of the control circuit, and an amplification circuit for turning on the SCR. In the operation of the control circuit, when delay time information is input through the keyboard, the information is transmitted to the microprocessor, and another signal is output by a predetermined program. The signal is weak to turn on the SCR, so a signal amplified by amplifying current and voltage using a high-speed switching transistor turns on the SCR and then precisely and sequentially turns on SCR1˜SCR3 up to 1 μm.

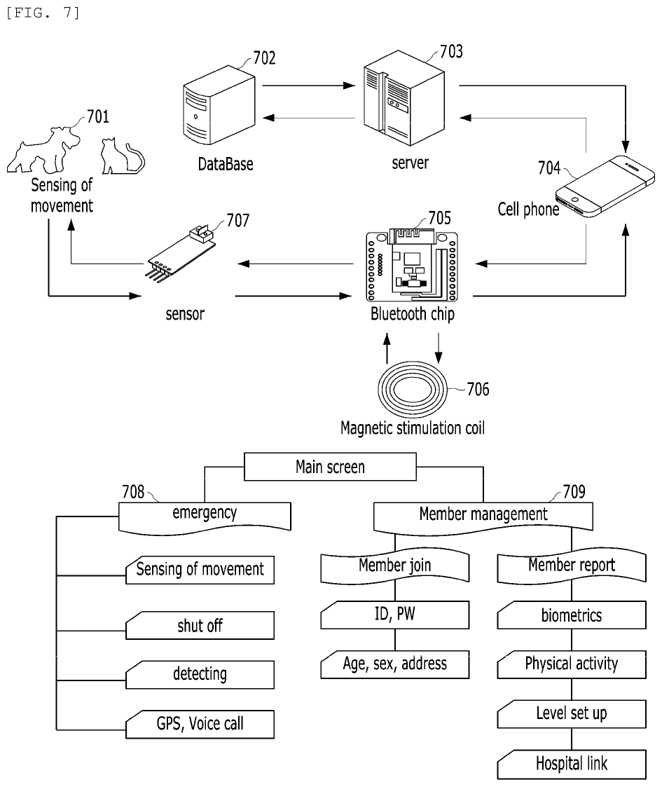

FIGS. 7 to 9 are views showing a process for treatment, communication, control, and connection with an associated person.

First, in order to handle an emergency such as musculoskeletal and shingle pain treatment using a CCD-CMOS sensor and a skin and electromyogram sensor and manage a quick treatment performance through a cell phone, for an animal patient of reference number 701, a method according to an embodiment of the present invention reduces economic, bodily, and mental burdens and transmits database data sent and accumulated from the sensors to reference numeral 702, thereby being able to counsel the patient, perform treatment, and receive special services through connection with facilities. Treatment is performed and locations are tracked using the sensors and the cell phone, and bio information and data are collected using the database, the cell phone, and the sensors and are transmitted to a medical facility through a wire/wireless system, so doctors, specialists, and guardians participate in treatment and cooperatively provide services, and uploading, feedback, disease study, and applications of a patient management system are implemented.

The apparatus proposed in an embodiment of the present invention is a system that is not limited by place and time, provides various monitoring areas, sets locations, accesses the server of reference numeral 703 through a wire/wireless communication network, and transmits a lesion state and location information to a cell phone of reference numeral 704 of a guardian registered on a server together with information stored in the database when it goes out of the area. Bluetooth of reference numeral 705 is sensed by mounting the CCD-CMOS sensor and skin and electromyogram sensor of reference numeral 707, it is changed into an activity suitable for an emergency, and monitoring may be added to a situation that may occur by adding GPS and voice recognition functions to the cell phone.

When a disease is traced and diagnosed in the GUI (graphic user interface) type of reference numeral 708, a data system is constructed when a problem is generated in a lesion for emergency treatment, member information is stored in a database when and ID and a password are input from an external special organization, a member management monitor, and a cell phone to a server, the homepage of the external organization is accessed using Urlparse, muscle skeleton, animal shingle, and a communication method of a pain treatment apparatus and a cell phone through a treatment coil of reference numeral 706 that can transmit connection information with a guardian and a medical facility using a telephone number access technology is shown.

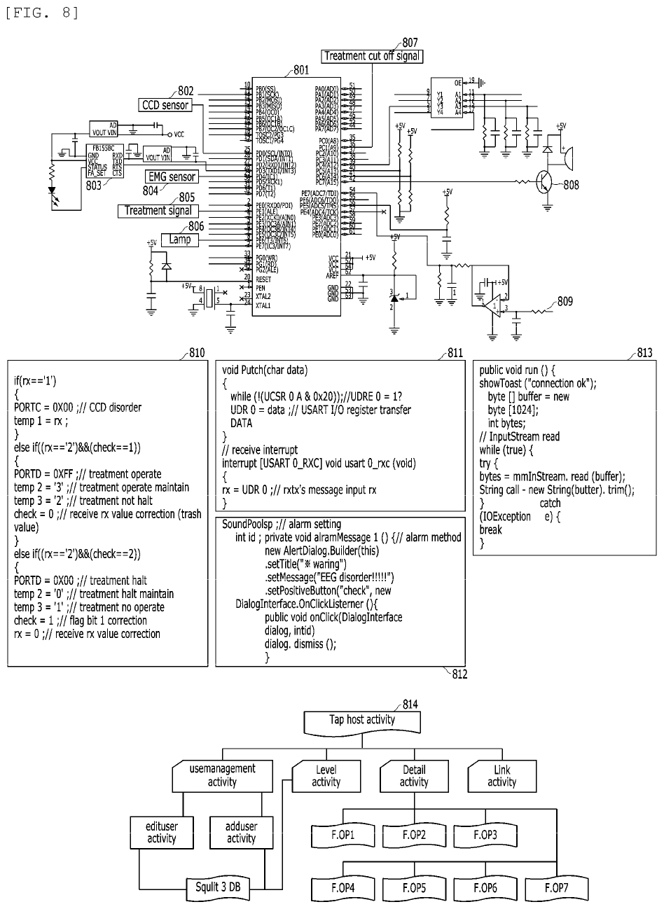

Referring to FIG. 8, according to the operation environment, a microprocessor of reference numeral 801 was operated by cooperation of a CCD-CMOS sensor of reference numeral 802, a skin, a conductivity sensor of reference numeral 804, etc., MSP430 and AVR series can be used, ATmega128 was used in the present invention, seven ports (A-G) are provided, 8 bits excluding the G port can be used, broadcast and URL that are open sources are used in response to signal of ATmega128, a homepage is liked, and a method of making an emergency phone call can be used in an emergency, which is difficult to bear, using ACT CALL. According to the operation principle, when there is a problem in activity and there is no motion, LEDs are turned on, when a danger is sensed through a sensor of reference numeral 809 using an application of a cell phone, the danger is recognized, the LEDs are turned on, and when a warning signal of reference numeral 808, sensor, etc. are sensed by a cell phone application through Bluetooth of reference numeral 803, a concerned persons are informed, whereby bidirectional communication is possible through a remote operation using a web and a phone application.

In Bluetooth, when data of sensor reach a USART I/O register, a reception interrupt is called, stored in a reception message RX, and transmitted/received to/from a cell phone, a received signal of a sensor module is input to ATmega128, an input value and a signals remotely controlled by a cell phone and a sensor are converted into digital signals and stored and analyzed in the reception RX, and then output values are output.

5V and 30˜50 mA are applied to Vcc of reference numeral 803, ATmega128 is connected, 7 and 8 pins are used for transmission/reception, Status is an On/Off pin, Tx of the microprocessor is connected to the RX, a received analog signal of the sensor module is converted into a digital signal, a 1-bit character is transmitted, an ADC value of the RX is read out, a light is turned on when a problem of reference number 810 is sensed, communication of an alarm code and Bluetooth is performed in 1 bit through a USART I/O register, an interrupt is used, is it stored in a message form in the RX, a code of reference numeral 811 is transmitted and received after a problem of the sensor is sensed, the sensor module call a timer from a source code stored in Atmega128, uses a Bluetooth module for a predetermined time (Test time: 5 Min), and transmits an emergency code to the cell phone, reference numeral 812 sends an emergency alarming code to the cell phone, an application informs reference numeral 813 of the problem with the sensor, and a musculoskeletal treatment, animal shingle, and pain treatment apparatus of reference numeral 805 and 807 is operated to performed treatment and then stopped.

When a signal of Bluetooth is received, it is changed into activity that is an execution unit configuring a screen, an interface is implemented, a server socket is generated, Bluetooth search is executed when a connection request is sensed, whether there is connected pairing, a client that is an important part is connected, a socket is generated, transmission/reception is performed through the socket, and a listening socket is closed and a thread is ended when socket generation fails.

Next, an input/output stream is set, a UUID protocol is used, a code configuring an environment that can communicate with Bluetooth is set, activity configuration is developed using Java and Xml in an android platform in application design, the OS of a cell phone uses Android, an application production method uses Eclipse and JDK, the version is 2.3.3 Gingerbread, the development environment is a Jetty server and an Elasticsearch database system, and a DB uses Elastic Search and database SQLite having Android as a search engine.

According to the application structure, reference numeral 814 for five services of a CCD sensor, muscle conductor, operation sensing, GPS, and emergency phone call that are performed after a signal is transmitted to a cell phone through Bluetooth, and diagnosis and management information is configured by eight activities, there is an activity configuration in the previous page, it is implemented in a server in HostActivity, log-in and a member information storage method are implemented in UserManagement, Tomcat that is an existing server requires many settings, a simple Jetty built-in server and Java language are used, and cell phone application is not operated when an emergency application requested by a client insets a corresponding right to an Manifast.xml file in an actual operation and then does not designate the right.

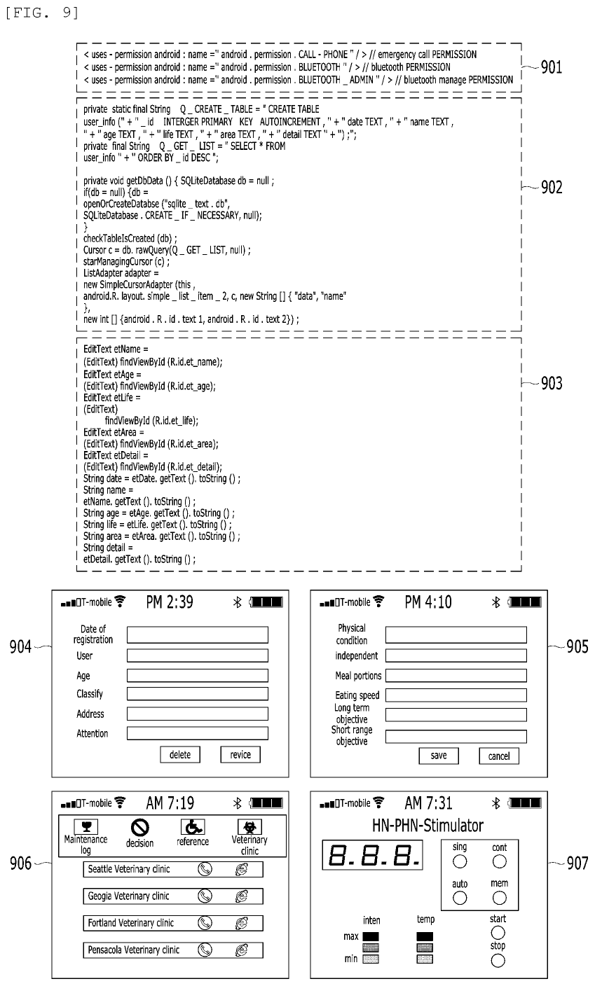

Referring to FIG. 9, for creating a database, a use log-in button and a member jointing button are made using an Oncreate method of reference numeral 901, EditText such as an ID, a password, an age, an address, a house type, a daily life, and life information is made, repetition of IID and password information is checked using a SetOnClicklistener method, a DB database of reference numeral 902 is created, a code configures a table, a table User_info is created, user IDs are automatically increased, a test type key value is added, a table value and a key value are received from Sqlite_test, and a reference numeral 903 receives a list of Key values from EditText as a code in a process of receiving and updating data in a table created by receiving user input, stores it as a string value, and then stores it in a DB file.

According to a result of implementing a mobile application, life and healthcare information are collected and recognized with concerned persons and guardian for early diagnosis and treatment through reference numeral 904, and can be referred to for external evaluation indexes such as neurology, BPSD, daily life activity, and performance ability, information can be supported from external instructions, lesion progression is continuously monitored, and it is possible to safely cope with an emergency when an emergency occurs. Further, when a user name, a house type, a name, an age, and a dwelling area are input and stored in an image implemented by data collection and support by a diagnosis support function, a diagnosis history image of an external medical organization shows up, and weekly and monthly collected data are data to be used for diagnosis and treatment management, whereby it is possible to diagnose shingle and verify diagnosis of pain treatment after shingle.

According to the image implemented in reference numeral 905, family members or guardians can periodically input symptoms and performance abilities that are shown in daily life, and can evaluate activities, mental, meals, excretion, bathing, wearing, cleanness, etc. in levels (1˜5) on the basis of observed results, diagnosis evaluation is downloaded as an external index and referred to for level determination when observation input such as eating activity, the amount of meal, a speed, and the degree of independence is finished, and information about alternative mediation treatment such as treatment, medicinal treatment, correction support, and whether facility program support is provided is provided through short and long-term objects. Further, according to an image of a connected treatment support of reference numeral 906, when in-house treatment is difficult I accordance with a management support function and a level determination result by observation, it is possible to provide a help for smooth treatment by connecting patient information to a close animal hospital or a special clinic, and appropriate mediation and connected treatment based of determination by a behavior and BPSD doctor are proposed. Reference numeral 907 performs early treatment by setting treatment in accordance with diagnosis and situations through communication between a cell phone and an apparatus.

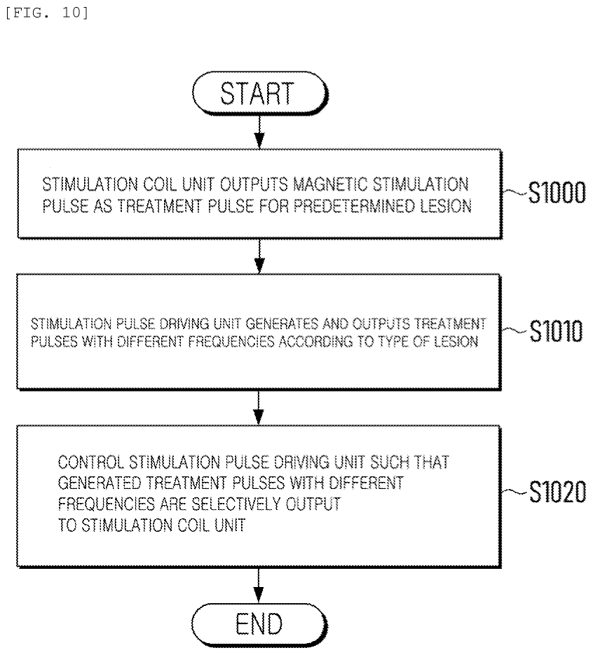

FIG. 10 is a flowchart showing a driving process of an animal musculoskeletal treatment apparatus according to an embodiment of the present invention.

Referring to FIG. 10 with FIG. 1 for the convenience of description, the animal musculoskeletal treatment apparatus 90 according to an embodiment of the present invention outputs a magnetic stimulation pulse as a treatment pulse for a predetermined lesion (S1000). The output magnetic stimulation pulse may be output as a sequence type pulse in a multiple discharge type.

A stimulation pulse driving unit of the animal musculoskeletal treatment apparatus 90 generates treatment pulses with different frequencies according to the types of lesions and provides the treatment pulses to a stimulation coil unit that outputs treatment pulses (S1010). The lesion may include a skin, muscle power, a ligament, and a skeleton, and a high-frequency treatment pulse may be provided. The type of a lesion may be selected on the basis of an instruction from a user, but may be measured and automatically selected by a sensor, etc., so it is not limited to any one specific type in an embodiment of the present invention.

Further, the animal musculoskeletal treatment apparatus 90 controls the stimulation pulse driving unit such that the generated treatment pulses with different frequencies are selectively output to the stimulation coil unit (S1020). In this process, the animal musculoskeletal treatment apparatus 90 can measure electromyogram through an EMG sensor and can adjust the treatment pulses on the basis of the electromyogram, in which the intensity, shape, and width of the treatment pulses can be adjusted.

Other than the above description, the animal musculoskeletal treatment apparatus 90 can perform various operations, and other details were sufficiently described above and refer to the above description.

Even through all components of embodiments of the present invention are combined in one unit or operated in combination in the above description, the present invention is not limited thereto. That is, the all components may be selectively combined and operated within the scope of the present invention. Further, although all the components may be implemented as individual hardware, some or all of the components may be selectively combined as computer programs having program modules that perform some or all of functions combined in one or several items of hardware. Codes and code segments of the computer programs may be easily inferred by those skilled in the art. The computer programs may be stored in a nontransitory computer readable media and read out and executed by a computer, thereby achieving embodiments of the present invention.

The nontransitory computer readable media is not a media that stores data for a short time such as a register, a cache, and a memory, but a media that can semipermanently store data and can be read out by a device. In detail, the programs may be stored and provided in a nontransitory computer readable media such as a CD, a DVD, a hard disk, a blueray disc, a USB, a memory card, and a ROM.

Although exemplary embodiments of the present invention were illustrated and described above, the present invention is not limited to the specific exemplary embodiments and may be modified in various ways by those skilled in the art without departing from the scope of the present invention described in claims, and the modified examples should not be construed independently from the spirit of the scope of the present invention.

What is claimed is:

1. An animal musculoskeletal treatment apparatus comprising:
a stimulation coil unit outputting a magnetic stimulation pulse as a treatment pulse for a predetermined lesion;
a stimulation pulse driving unit generating and providing, to the stimulation coil unit, treatment pulses with different frequencies according to a type of a lesion; and
a controller controlling the stimulation pulse driving unit such that the generated treatment pulses with different frequencies are selectively output to the stimulation coil unit.

2. The animal musculoskeletal treatment apparatus of claim 1, further comprising an electromyogram sensor,
wherein the controller controls the stimulation pulse driving unit to generate a treatment pulse for a predetermined lesion further in consideration of a sensing value of the electromyogram sensor.

3. The animal musculoskeletal treatment apparatus of claim 1, wherein the stimulation pulse driving unit uses a multiple discharge method of outputting treatment pulses by sequentially generating pulses in a pulse sequence type for a predetermined lesion.

4. The animal musculoskeletal treatment apparatus of claim 3, wherein the stimulation pulse driving unit outputs a treatment pulse by adjusting pulse intensity, a pulse shape, and a pulse width in accordance with a delay time on the basis of the multiple discharge method.

5. The animal musculoskeletal treatment apparatus of claim 1, wherein the stimulation pulse driving unit configures a pulse forming circuit composed of a resistor, a capacitance, and an inductance to supply energy to the stimulation coil unit, and the pulse forming circuit configures a multiple L-C network.

6. A method of driving an animal musculoskeletal treatment apparatus, the method comprising:
outputting a magnetic stimulation pulse as a treatment pulse for a predetermined lesion by means of a stimulation coil unit;
generating and providing, to the stimulation coil unit by means of a stimulation pulse driving unit, treatment pulses with different frequencies according to a type of a lesion; and
controlling the stimulation pulse driving unit such that the generated treatment pulses with different frequencies are selectively output to the stimulation coil unit by means of a controller.

7. The method of claim 6, further comprising measuring a state of a muscle by means of an electromyogram sensor,
wherein the controlling controls the stimulation pulse driving unit to generate a treatment pulse for a predetermined lesion further in consideration of a sensing value of the electromyogram sensor.

8. The method of claim 6, wherein the providing of treatment pulses to the stimulation coil unit uses a multiple discharge method of outputting treatment pulses by sequentially generating pulses in a pulse sequence type for a predetermined lesion.

9. The method of claim 8, wherein the providing of treatment pulses to the stimulation coil unit outputs a treatment pulse by adjusting pulse intensity, a pulse shape, and a pulse width in accordance with a delay time on the basis of the multiple discharge method.

10. The method of claim 6, wherein the stimulation pulse driving unit configures a pulse forming circuit composed of a resistor, a capacitance, and an inductance to supply energy to the stimulation coil unit, and the pulse forming circuit configures a multiple L-C network.
